(19) United States
(12) Patent Application Publication
(10) Pub. No.: US 2026/0360519 A1
(43) Pub. Date: Nov. 12, 2026

(54) INTELLIGENT SYSTEM TO STREAMLINE COMMUNICATIONS AND REDUCE INTERRUPTIONS IN THE RADIOLOGY DEPARTMENT

(71) Applicant: KONINKLIJKE PHILIPS N.V., EINDHOVEN (NL)

(72) Inventors: Qianxi LI, CAMBRIDGE, MA (US); Victor WIJN, EINDHOVEN (NL); Merlijn SEVENSTER, HAARLEM (NL); Peter Jacobus HAIMA, EINDHOVEN (NL); Jaap KNOESTER, UTRECHT (NL); Vincentius Paulus BUIL, VELDHOVEN (NL); Marleen Johanna Jacoba VAN LEENGOED, WAALRE (NL); Mark Anthony HENNESSY, EERSEL (NL); Daan KUHLMANN, HEESCH (NL); Sandra VOSBERGEN, LIEMPDE (NL); Hildebrandus Johannes LAMB, LEIDEN (NL); Sandeep Madhukar DALAL, WINCHESTER, MA (US); Olga STAROBINETS, NEWTON, MA (US)

(21) Appl. No.: 19/124,928

(22) PCT Filed: Oct. 25, 2023
(86) PCT No.: PCT/EP2023/079819

§ 371 Date: Apr. 28, 2025

Related U.S. Application Data
(60) Provisional application No. 63/421,611, filed on Nov. 2, 2022.

(51) Int. Cl. G16H 40/20; G06F 3/0482; G16H 30/40; G16H 50/20

(52) U.S. Cl. CPC G16H 40/20; G06F 3/0482; G16H 30/40; G16H 50/20

(57) ABSTRACT

A non-transitory computer readable medium (16) stores instructions readable and executable by an electronic processor (14) to perform a method (100) of requesting radiology assistance. The method includes providing, on a display device (24) of a requestor electronic processing device (18) operable by a requestor, a requestor graphical user interface (GUI) (28); receiving one or more inputs from the requestor to fill in fields in the requestor GUI to generate an assistance request (40) for radiology assistance; transmitting the assistance request for radiology assistance to a receiver electronic processing device (30) operable by a radiologist; providing, on a display device (36) of the receiver electronic processing device, a receiver GUI (38) showing a list (44) of assistance requests for radiology assistance; and tracking and labelling a status of each assistance request for radiology assistance in the list.

### FIELD

The following relates generally to the radiology arts, radiology assistance arts, graphical user interface (GUI) generation arts, picture archiving and communication system (PACS) arts, and related arts.

### BACKGROUND

Radiologists are frequently interrupted by requests for radiology assistance during the radiologist's workday. Radiologists are interrupted between once every 4 to 12.1 mins during regular business hours and receive an average of 72 phone calls during a typical 12-hour overnight shift (see, e.g., Rachel M. Wynn, PhD, et al. The Impact of Interruptions on Chest Radiograph Interpretation Effects on Reading Time and Accuracy. Academic Radiology. VOLUME 25, ISSUE 12, P1515-1520 Dec. 1, 2018; Drew T, Williams L H, Aldred B, Heilbrun M E, Minoshima S. Quantifying the costs of interruption during diagnostic radiology interpretation using mobile eye-tracking glasses. J Med Imaging (Bellingham). 2018 July; 5(3):031406. doi: 10.1117/1.JMI.5.3.031406. Epub 2018 Mar. 2. PMID: 29531970; PMCID: PMC5833804). These interruptions are primarily in the form of answering phone calls, responding to pages, and provide case consultants in-person. Interruptions can lead to increased reading times, introduce patient safety concerns during reads of abnormal cases, increased stress, or frustration for the radiologist. In addition, many questions are actually not that urgent, and can wait a couple of minutes, this is often also the time needed for the radiologist to finalize their current task.

Questions requesting radiology assistance are mainly raised by referral physicians and technologists. Interruptions from referral physicians mostly relate to study review, order questions, and asking for study results, while imaging technologists often request radiology assistance in protocol assignment, image check, questions for findings, etc.

Technologists or referral physicians could also be disrupted when sending or receiving a response through phone calls, and thus asynchronous communication through text-based messaging is preferred in most situations. The time to require a reply depends on the urgency of the task the receiver is executing and the urgency of the question. Not having an answer can affect the patient (clinical urgency) or can disrupt the workflow. Complaints about a delay in response or difficulties in reaching the available radiologist(s) at certain critical moments are common. Currently, interruptions (either via phone call or page/text message) do not distinguish between matters that are urgent and those that are not.

Depending on the setup of each clinical institution, there could be one or multiple radiologists assigned to reply to the questions or requests for radiology assistance raised during a given time. The most common radiologist(s) assignments in a day shift are based on modality or specialty section of the study on a rotation basis. In such a setting, radiologists would only be responsible for questions raised from the assigned sections or modalities. On a weekend or nightshift, there might be only one on-call radiologist available. And thus, the radiologist would be responsible for answering all questions raised from any section or modality. In addition, multiple radiologists may be available at larger medical facilities or hospitals, and thus will be responsible for a larger part of the hospital or hospital group.

Moreover, requests for assistance from the on-call radiologist vary in urgency, dependent for example on whether the patient is still in the scanner or the dressing room, the type and severity of the case (clinical urgency), and the patient history. Sometimes a request change in urgency over time, or even become moot because an answer has been found in another way, or because the patient had to leave due to another appointment. Sometimes the radiologist is unable to take a call when the requesting technician or physician would like to reach out to them, and the other way around. When responding to a request for assistance, the radiologist is completely drawn out of the image review or examination reading that the radiologist was doing. In some cases, it even occurs that the radiologist is managing several phone calls at the same time while assisting urgent requests. This can lead up to very stressful working days, and inefficient communication and collaboration.

A radiologist may be able to note outcomes immediately via a chat function directly with the appropriate stakeholder(s) assigned to that specific case ticket. In practice though, requests for assistance still result in radiologists and technicians interrupting and calling each other sometimes because of the benefits of verbal communication. However, the interruptions can happen at unfortunate moments, often resulting in a disruption of workflow, an increase in cognitive load, frustration, stress and generally a lot of chaos during a workday.

The following discloses certain improvements to overcome these problems and others.

### SUMMARY

In some embodiments disclosed herein, a non-transitory computer readable medium stores instructions readable and executable by an electronic processor to perform a method of requesting radiology assistance. The method includes providing, on a display device of a requestor electronic processing device operable by a requestor, a requestor graphical user interface (GUI); receiving one or more inputs from the requestor to fill in fields in the requestor GUI to generate an assistance request for radiology assistance; transmitting the assistance request for radiology assistance to a receiver electronic processing device operable by a radiologist; providing, on a display device of the receiver electronic processing device, a receiver GUI showing a list of assistance requests for radiology assistance; and tracking and labelling a status of each assistance request for radiology assistance in the list.

In some embodiments disclosed herein, an assistance request method includes receiving one or more inputs from a requestor to fill in fields in a requestor GUI displayed on a display device of a requestor electronic processing device operable by a requestor to generate an assistance request for radiology assistance; transmitting the assistance request for radiology assistance to a receiver electronic processing device operable by a radiologist; and tracking and labelling a status of the assistance request for radiology assistance on a receiver GUI provided on a display device of the receiver electronic processing device.

In some embodiments disclosed herein, an assistance request method includes determining an urgency of a medical imaging examination performed by a requestor; determining an interruption score of a radiologist from whom assistance is being requested by the requestor; and determining at least one follow-up action for the radiologist to provide to the requestor based on the determined urgency and the determined interruption score.

One advantage resides in reducing interruptions for radiologists during an imaging workflow.

Another advantage resides in reduced task switching between radiologists during imaging examination workflows.

Another advantage resides in increased quality of radiologist workflows by reducing interruptions during the workflows.

Another advantage resides in reducing complaints in delayed responses for radiologists during an imaging workflow.

Another advantage resides more easily identifying a radiologist who requests assistance during an imaging workflow.

Another advantage resides in automated creation, prioritization, and tracking of assistance requests.

Another advantage resides in enabling radiologists to maintain focus and minimize the impact of distractions by others.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an assistance request apparatus in accordance with the present disclosure.

FIG. 2 diagrammatically illustrates an assistance request method using the apparatus of FIG. 1.

FIG. 3 diagrammatically illustrates electronic processing devices of the apparatus of FIG. 1 operable by different users.

FIG. 4 diagrammatically illustrates another embodiment of an assistance request apparatus in accordance with the present disclosure.

### DETAILED DESCRIPTION

Presently, imaging technicians and physicians requesting assistance from a radiologist primarily use synchronous communication pathways such as telephone or videocall (e.g., some institutions use Zoom, Skype, or Microsoft Teams) or pagers. Such requests are also usually not prioritized, and so the radiologist is frequently interrupted to handle radiology assistance requests that may be of low priority or can wait.

The following provides a system for providing asynchronous radiology assistance request handling with assigned request priorities. A requestor graphical user interface (GUI) is provided via which a requestor can submit a request for radiology assistance. The request for radiology assistance may for example come from an imaging technician requesting assistance in setting up an imaging scan or so forth, or from a doctor or other medical professional requesting assistance in understanding a radiology image or content of a radiology report. The requestor GUI includes a field for assigning a priority on a predefined prioritization schedule, for example with several discrete priority levels ranging from “not urgent” to “stat.” A timeframe for the reply and details on the effect on the workflow and/or the patient (e.g., “a patient is positioned on the table) to provide the radiologist with the information to conclude that not answering the question will disrupt the workflow, may also be specified by the user. It could be however, that the radiologist has a clinical emergency, in that case they will always prioritize the request over the workflow interruption. The requestor GUI also allows the requestor to attached documentation or links to documentation, such as a link to the subject examination in a Picture Archiving and Communication System (PACS) for example. The requestor GUI may also permit flexible addressing options, such as specifying a specific radiologist as the addressed receiver, or specifying a role (e.g., receiver can be any radiologist with expertise in a particular imaging modality), or no specific addressee specified. A module then handles transmission of the request to a suitable radiologist. In addressing other than a specific radiologist the system then assigns the receiving radiologist based on who is on-call, their expertise and workload.

At the receiving radiologist end, a receiver GUI is provided with a worklist of the requests addressed to that receiving radiologist, organized by priority, specified reply-by time, or other criteria. The receiver GUI also provides communication pathways such as a videoconferencing pathway, text messaging pathway, VOIP telephonic pathway, and/or so forth via which the receiving radiologist can provide a suitable reply. At the requestor GUI, receiver GUI, or both, each request may also be labeled or updated as to status, e.g. “Not sent,” “New,” “In progress,” “Re-opened,” “closed,” et cetera. In some examples, the worklist can include questions addressed to a section/role, in that case they can pick up the cases based on their availability and their own process. In that case the requestor will also need to know which ones are taken up already, by whom etc.

In some embodiments disclosed herein, the requestor can attach one or more clinical images or image series that are annotated by the requestor to point out an image feature or other image content which is the subject of the request (e.g., an arrow pointing to an image feature for which a more specific interpretation is being requested). As it is undesirable to annotate the images in the PACS in this way, an annotated copy of the image, with possibly reduced resolution and/or which is compressed, may be attached to the request. If the attached image is of reduced resolution or is compressed, then a link to the original image in the PACS is preferably provided. For example, clicking on the attached image using a mouse or other user input device may retrieve and display the original image.

In some variant embodiments disclosed herein, in addition to providing asynchronous radiology assistance request handling for requests originating with imaging technicians or other non-radiologist medical personnel, the system may further provide asynchronous radiology assistance request handling for radiologist to radiologist messaging.

In some embodiments disclosed herein, artificial intelligence (AI) components can be integrated into the system. For example, if an imaging system is set up with computer-aided diagnostic (CADx) modules, then if a CADx module detects a suspicious feature possibly being an incidental finding it may automatically generate a request for a radiologist to confirm or reject the conclusion of the CADx, or to take other action such as acquiring additional images of the suspicious feature. Similarly, automated detection of an image quality issue by an AI module could trigger an automatic request. The urgency of a request could also be set automatically rather than manually, based on machine learned (ML) analysis of the content of the request for example.

In other embodiments disclosed herein, the system could monitor upcoming radiology examinations to ensure that all required input from the radiologist is completed ahead of the start of the exam. For example, if the radiologist has been asked to specify imaging sequences for the exam card, then if this is not done within some set time before the exam (e.g., at least 24 hours ahead) then a request to do so is automatically generated.

In other embodiments disclosed herein, the ticket priority and/or the recommended mode of communication (e.g. telephonic, chat, video with screen-sharing, or so forth) is monitored and adjusted in real-time based on real-time feedback such as monitoring eye movements of the radiologist to assess the radiologist's engagement in his or her current work (e.g. a radiology examination reading), the complexity of the radiology examination currently being read by the radiologist, the patient location (which can change over time), and so forth. The technician's request urgency and the cost of interruption for the radiologist are assessed in real-time and balanced to update the request priority and/or recommended mode of communication in real time.

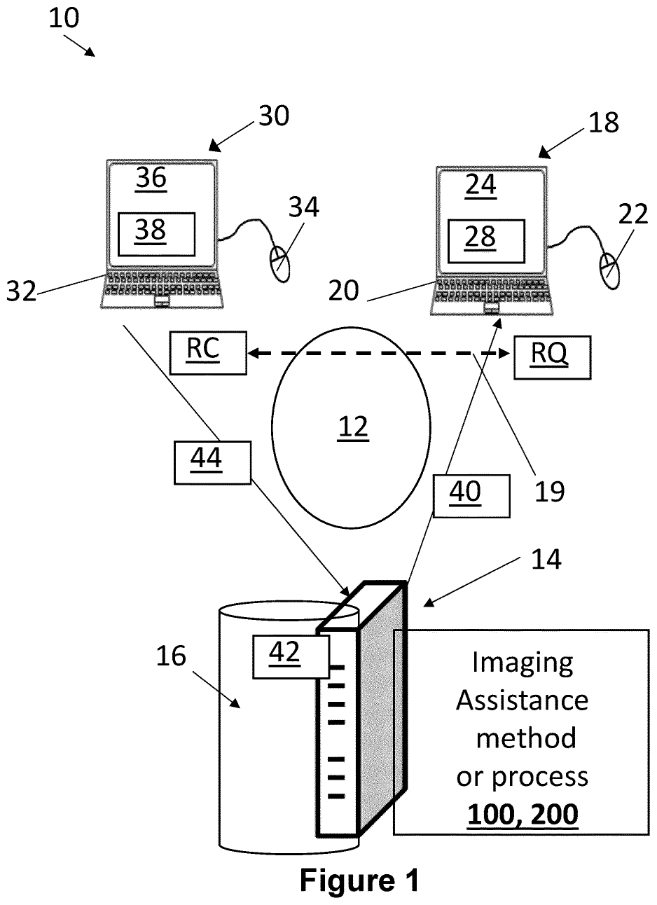

With reference to FIG. 1, an illustrative apparatus 10 for providing assistance during imaging examinations is shown. As shown in FIG. 1, the monitoring apparatus 10 includes, or is accessible by, a server computer 14. The server computer 14 comprises a computer or other programmable electronic device that includes or has operative access to a non-transitory computer readable medium. The server computer 14 is connected with other devices over a communications network 12, such as the Internet, optionally along with one or more local area networks (LAN) or wide-area networks (WAN), such as a LAN or WAN of the hospital at which the PACS 12 is located, and/or a LAN or WAN of the vendor or of a cloud computing service provider providing the server computer 14 to the vendor. It will be appreciated that the server computer 14 could be implemented as a plurality of server computers, e.g., interconnected to form a server cluster, cloud computing resource, or so forth, to perform more complex computational tasks.

A requestor electronic processing device 18, such as a workstation computer, or more generally a computer, a mobile device (e.g., a tablet computer), is operable by a requestor (RQ), (such as a technologist performing an imaging examination or the like) to provide a user interface with an imaging assistance method or process 100 running on the server computer 14. The requestor electronic processing device 18 includes typical components for a user interfacing computer, such as an electronic processor 20 (e.g., a microprocessor), at least one user input device (e.g., a mouse, a keyboard, a trackball, and/or the like) 22, and a display device 24 (e.g., an LCD display, plasma display, and/or so forth). In some embodiments, the display device 24 can be a separate component from the requestor electronic processing device 18 or may include two or more display devices.

A receiver electronic processing device 30, such as a workstation computer, or more generally a computer, a mobile device (e.g., a tablet computer), is operable by a receiver (RC), (such as a technologist monitoring one or more imaging examinations or the like) to provide a user interface with an imaging assistance method or process 100 running on the server computer 14. The receiver electronic processing device 30 includes typical components for a user interfacing computer, such as an electronic processor 32 (e.g., a microprocessor), at least one user input device (e.g., a mouse, a keyboard, a trackball, a dictation device, and/or the like) 34, and a display device 36 (e.g., an LCD display, plasma display, and/or so forth). In some embodiments, the display device 36 can be a separate component from the receiver electronic processing device 30 or may include two or more display devices.

The server computer 14 includes a database comprising a non-transitory storage media 16 which may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 16 herein is to be broadly construed as encompassing a single medium or multiple media of the same or different types. Likewise, the electronic processor 20 may be embodied as a single electronic processor or as two or more electronic processors. The non-transitory storage media 16 stores instructions executable by the at least one electronic processor 20. The instructions include instructions to generate a visualization of a requestor graphical user interface (GUI) 28 for display on the display device 24 of the requestor electronic processing device 18, and a visualization of a receiver GUI 38 for display on the display device 36 of the receiver electronic processing device 30.

The apparatus 10 is configured as described above to perform an imaging assistance method or process 100. The database 16 stores instructions executable by the server computer 14 (and/or by the requestor electronic processing device 18 and/or the receiver electronic processing device 36) to perform the imaging assistance method or process 100. In some examples, the method 100 may be performed at least in part by cloud processing (that is, the server computer 16 may be implemented as a cloud computing resource comprising an ad hoc network of server computers).

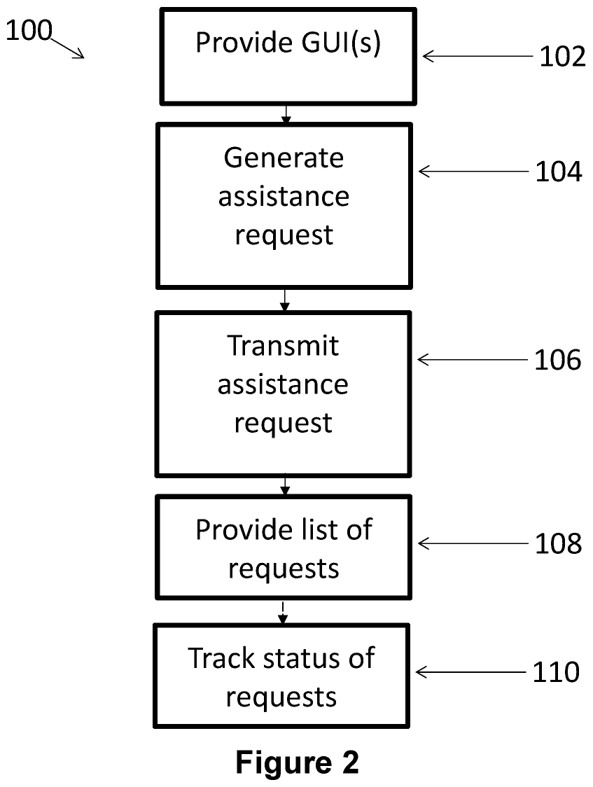

With reference to FIG. 2, and with continuing reference to FIG. 1, an illustrative embodiment of an instance of the imaging assistance method 100 is diagrammatically shown as a flowchart.

At an operation 102, the requestor GUI 28 is provided on the display device 24 of the requestor electronic processing device 18. The requestor GUI 28 can include one or more fields via which the requestor RQ can provide inputs via the at least one user input device 22. Optionally, at this point, the receiver GUI 38 can be provided on the display device 36 of the receiver electronic processing device 30.

At an operation 104, one or more inputs from the requestor RQ can be received to fill in fields in the requestor GUI 28 to generate an assistance request 40. In one example, a field of the requestor GUI 28 includes an addressee field configured to receive an addressee as any one of: (i) a specific radiologist; (ii) a radiologist role; or (iii) no addressee specified. In another example, a field of the requestor GUI 28 includes a field for assigning a priority on a predefined prioritization schedule. In another example, a field of the requestor GUI 28 includes a field for attached documentation or links to documentation. In another example, a field of the requestor GUI 28 includes a field for requesting a specific radiologist for assistance with the assistance request 40. In another example, a field of the requestor GUI 28 includes a field for attaching a clinical image that is the subject of the assistance request 40. In this example, the requestor GUI 28 further provides a field via which the requestor RQ can annotate the clinical image, and the annotated clinical image is entered into the field for attaching the clinical image. In yet another example, the requestor GUI 28 includes a field for updating the status of the assistance request 40. In yet another example, the requestor GUI 28 includes a field for displaying a status of an imaging acquisition or workflow. These are merely examples and should not be construed as limiting.

In some embodiments, the assistance request generation operation 40 can be performed using an artificial intelligence (AI) component 42 (stored in the database 16). For example, the AI component 42 can comprise a multi-classification machine learning model. In one example, the AI component 42 is configured to analyze a computer-aided diagnostic (CADx) feature of an imaging device used in an imaging examination performed by the requestor RQ. The assistance request 40 is generated based on the analysis of the CADx feature. In another example, the AI component 42 is configured to analyze a clinical image generated by an imaging device used in an imaging examination performed by the requestor RQ. The assistance request 40 is generated based on the analysis of the clinical image. These are merely examples and should not be construed as limiting.

Once generated, the assistance request 40 is transmitted to the server computer 14. At an operation 106, the assistance request 40 is transmitted to the receiver electronic processing device 30. At an operation 108, a list 44 of assistance requests 40 is provided on the receiver GUI 38. The list 44 of assistance requests 40 displayed on the receiver GUI 38 includes a worklist of the requests addressed to the receiver RC based on one or more criterion (e.g., priority, reply-by-time, and so forth). In addition, the receiver GUI 38 includes one or more fields via which the receiver RC can provide inputs via the at least one user input device 34. The field(s) of the receiver GUI 38 can include, for example, one or more of a field for a communication pathway 19 between the receiver and each requestor who submitted the assistance request 40 (which can then be established), a field for updating the status of the assistance request 40, and so forth.

At an operation 110, each assistance request 40 on the list 44 can be tracked and labeled (“Not sent”, “New”, “In progress”, “Re-opened”, “closed”, etc.).

In some embodiments, the method 100 can include monitoring a schedule of upcoming radiology examinations including detecting an upcoming radiology examination with an incomplete examination card that requires information from a radiologist (i.e., the requestor RQ) to complete the examination card. A request can be generated for the requestor RQ to provide the information required to complete the examination card before a beginning of an imaging examination.

### Example

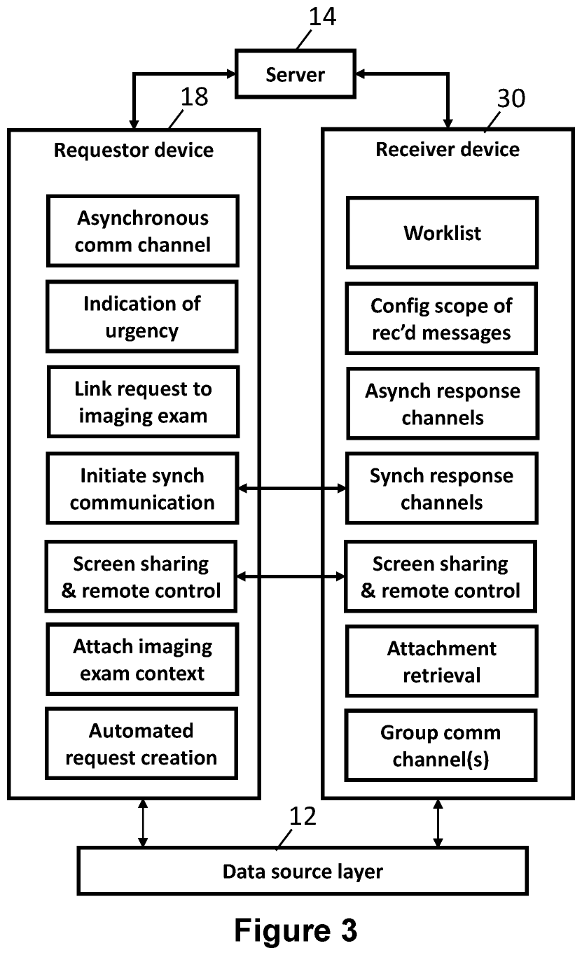

The following describes the apparatus 10 and the method 100 in more detail. FIG. 3 diagrammatically depicts aspects of this example, including modules implemented by the requestor device 18, the receiver device 30, and the server 14. The server computer 14 can comprise one or more modules to perform the method 100 along with various modules of the requestor and receiver devices 18 and 30. For example, as shown in FIG. 3 one of the modules can include a module (e.g., implemented at the requestor device 18) to initiate communication requests. The module is for users to send a communication request. The main users of the requestor device 18 may include technologists or referral physicians, and less frequent users may include medical residents and radiologists. The user of the receiver device 30 is typically a radiologist. Both asynchronous and synchronous channels can be used to create a communication request for radiology assistance. In this illustrative example, various features are also supported to streamline communication requests, including an indication of urgency, linking requests with a study, screen-sharing, attaching study or patient context, and enabling omnichannel (i.e., automated) generate requests, for example initiated by AI modules, managing requests status, and sending a reminder.

The main portion of a communication request is the content. The content usually contains the questions or requests raised for a scan or study. The content of the communication requests is conveyed in a message in asynchronous communication and could be sent through a ticket or chat function. In synchronous communication, the content can be sent through phone calls, web-calls, or video calls. Also, screen-sharing and remote control are enabled to augment the content. Screen-sharing and remote control are enabled to allow the receivers to have access and control over to the scanner from a remote setting.

The question can be described in free text or using canned, pre-defined questions that can be selected by clicking a graphical UI element (e.g., tile). It is also desirable to indicate the urgency of the request and identifiers to link the request to a study along with content. The urgency of the request defined the timespan within which the request needs to be answered. Input for the urgency of the request can be generated in multiple ways, including but not limited to through user input or AI-based prediction. The urgency can be indicated in different ways, for instance using a scale of items (e.g., not urgent-urgent-very urgent-STAT). Alternatively, the urgency is reflected using targeted response time in minutes, e.g., within 30 min. In this manner, the urgency can be manually set or selected from a list of pre-defined target response times (e.g., within 0 minutes, within 5 minutes). In addition, workflow information (e.g., is there a patient on the table, when is the missing protocol needed (patient scheduled)), etc.) can be included with the determined urgency.

In some embodiments, the urgency is determined by the AI component 42. For example, a multi-classification machine learning model can be trained to predict the urgency level of the request. The input of the model would be the content of the question, characteristics of the patient, and study, such as modality, department, scan type, etc. The training label or output would be the timespan in categorical format within with a response should be provided, e.g., ‘within 5 mins’, ‘within 30 mins’, ‘within 1 hour’ etc. The machine learning model can also factor in the actual response times (e.g., duration of time interval from request submitted until request opened) as alternative ground truth.

It is also sometimes beneficial to link the communication requests with a study to provide study context, such as the identifier of the study should be provided. This can be realized in multiple ways. Senders can manually input the study identifier. Alternatively, a worklist of the studies could be provided for the senders to select and associated the request with. The worklist is based on patients that are being scanned or will be scanned today. This list can be compiled based on information that is contained by the scanners (and extracted using console extraction techniques), HL7 messaging the Radiology Information System (RIS), the Electronic Medical Record (EMR) database. For example, the requestor and receiver devices 18 and 30 can be linked to such databases, which an option to select a patient and/or add further details to the request.

Another feature of some embodiments allows the sender to attach more patient and study context to the message, such as the DICOM images, patient history, referral notes, etc. This serves to provide complementary information to clarify or augment the context of the communication request, which improves the efficiency of communication. While providing this information as an attachment is useful to assist the receiver in understanding the clinical context of patient and study, the receiver can still access the study context through other channels, which are already enabled in the system. One alternative way is through the screen-sharing function. With the identifier provided, the receiver can also access study/patient information in other clinical informatics systems.

A communication request can be initiated through multiple channels. Another optional feature is to adopt an omnichannel (or multichannel) approach to allow communication requests to be generated from multiple sources, such as AI-generated requests. The request should link to the patient automatically, so provide all the patient details needed from (e.g., the RIS or PACs or EMR). There should be a link to retrieve the correct patient history at receiver site as well.

Through the requestor device 18, the requestor can send the communication request with patient information, question and urgency indication. Once sent, a message would be generated in the chat window opening up on the receiver device 30. The radiologist could follow up on the request through synchronous and asynchronous communication.

To facilitate automatic routing of the request for radiology assistance, a role-based log-in can be used to identify the users on both the sender's and receiver's side.

Another module can include an AI module to generate the communication request for radiology assistance. The module could contain multiple AI engines to predict different communication requests and/or to detect image features that warrant radiologist consultation. In one embodiment, the module receives the DICOM image and applies one or more AI engines implementing CADx analysis to detect findings that need urgent radiologist attention, e.g., pneumothorax, potential COVID-19 or aneurysm. In another embodiment, an AI module detects potential image quality issues (e.g., motion artefact) with the images (x-ray, CT, MR) or missing sequences (CT, MR). The output of the module provides the content of the communication request and would be used as input to generate communication requests containing all or part of the elements listed above (e.g., question, urgency). Each auto-generated request can be sent to a relevant requester (e.g., tech) for completing/editing the request if necessary and/or can be sent directly to corresponding receiver(s).

Another module can include a module to direct communication requests to corresponding receiver(s). The module directs the communication requests to the receiver, without the absolute need for the sender to indicate the individual that needs to pick up the request. The requestors initiate communication requests and the receivers who are responsible to reply to the requests.

Users of the system to send requests to individuals (e.g., Dr. Doe) or to roles (e.g., any radiologist that can help with MR brain scans). The system can be configured to support only communication directly with individuals or role-based communication or both.

Before directing the communication request, the scope of the request is identified. The scope refers to selected sections or modalities from which the receiver is responsible for answering the requests raised. The sender could indicate the scope of the request. The scope could also be identified if study context with modality or scanner information is provided. Alternatively, AI-models could be trained to automatically categorize the scope of the request and assign it to the most suitable receiver(s). For example, a natural language processing (NLP) model could be trained to extract information from the content of the request and predict the scope.

With the scope identified, a communication request can be directed in multiple ways. For example, the senders could specify the receiving individual radiologist, and the request for radiology assistance is then sent to that specific radiologist.

The system in some embodiments may automatically identify the appropriate receiving radiologist and direct the request for radiology assistance to that radiologist without the need for the sender to specify the receiver. This can be realized through role-based directing, in which case each radiologist would use log-in information to indicate the responsible scope of the requests. For example, the receiving radiologist can indicate that he/she is available to receive requests related to brain MR (or other clinically relevant scan types), in which case the system would then directs all questions regarding the brain scans to this account. When multiple radiologists are available to answer requests from the same scope, the system could allow multiple radiologists to log-in under the same role-based account. For example, under the account ‘rads for brain scans,’ multiple radiologists can log-in into the same system and assess the same list of requests directed.

Communication requests for radiology assistance can also be directed based on availability or workload.

When multiple receivers are available to answer requests from the same scope, the system could also intelligently direct the communication request based on the workload of each user in the same scope. If system detects that one radiologist is not available or is under heavy image interpretation workload, requests would be directed to other radiologists responsible for the scope. A Bayesian probability model, for example, could be trained to balance the workload and urgency of the request in order to maximize the efficiency and minimize interruptions. The output of the Bayesian probability model would be the probability of answering request within the given time span given the current workload.

The radiologists can configure the scope of the requests to be received. This would be useful in the night/weekend shift or similar situations when only one radiologist is available to answer all the requests. For example, during nightshifts, there is often a dedicated employee that can be called upon in case of too much workload/emergencies etc. The system 10 could maybe do this automatically or predict when this is necessary too.

A radiologist log-in may be adjusted according to the chosen routing mechanisms mentioned above. A radiologist can choose to log in through user-specific identification or through role-based log-in, in which radiologist would log in as the section or scanner for which he/she is supporting.

The receiver device 30 provides mechanisms for a radiologist to provide a response. The main user of the receiver device 30 is radiologists, although other users who are responsible for responding to communication requests for radiology assistance are contemplated.

At the receiver device 30, a comprehensive worklist presents an overview of the communication requests, which can be opened by the radiologist at his/her leisure, thus allowing the radiologist to plan for tasks and time assignments and thus reduce interruptions (e.g., by giving the radiologist a chance to finish a task that he/she has started, which reduces task-switching). For each request in the worklist, basic information, status, requestor and urgency level would be presented. Basic information helps to link the study with the requests. In some embodiments, minimal information to provide may be the study/patient identifier. Additional information could also be provided to provide a brief clinical context, such as modality, room, scanner technologists, scan type, patient information, etc. A request can have multiple statuses, including but not limited to “Not sent,” “New”, “In-progress”, “Closed,” “Re-opened,” etc. Filtering and re-ordering functions are also enabled for the radiologist to control and reorganize the worklist content. In addition, the receiver device 30 can forward or auto-forward the requests to the requestor device 18. One of the struggles of the radiologist is that they often get questions that are not really for them (i.e., unnecessary questions). Additionally, if they are being called away, the system should automatically reroute (particular more urgent) questions.

Each incoming request for radiology assistance has an urgency associated with it. In one embodiment, the urgency is indicated in expected response time in minutes. In this embodiment, the expected response time can be shown and/or the current waiting time and/or the expected response time minus the current waiting time. In this embodiment, a complex filter can be applied that is based on urgency level that promotes requests that may be non-urgent but are pushed to the top of the list because the waiting time exceeded the expected response time. In some examples, the AI component 42 can couple a waiting time with the workflow to see which request is at the top of the list or should be on top of the list.

In a suitable default approach, only requests for which the radiologists are responsible for replying would be sent, however in some embodiments the radiologist can be allowed to configure the scope of the requests to be received. Channels for asynchronous and synchronous communication, screen sharing, and remote control are supported. The receiver device 30 supports not only one-to-one communications, but in some embodiments also group communications across discipline, which serves the need for group consultation and multi-disciplinary meetings and learning for university hospitals.

The system can also manage the status of the communication and provide reminders when needed. This enables both the requestor and the receiver to automatically update the status of the communication and provide reminder when needed. A request for radiology assistance can have multiple statuses, including but not limited to “Not sent,” “New”, “In-progress”, “Closed,” “Re-opened,” etc. “Not sent” indicates that a communication request has been created but not sent to the receiver. New “indicates that has been sent to the receiver but no action has been taken on the communication request. Once a New ticket's status has been changed, it can never be set back to New. “In-progress” indicates that a communication request is under review by the receiver (i.e., for role-based requests, the request can include whom the requested party is). “Closed” indicates that a communication request has been answered or that the receiver is not responsible for answer. “Re-opened” indicates that a communication request has been closed before, but follow-up questions/request has been raised.

“Not sent,” “New”, “In-progress” are status generated by the system automatically. Users need to decide to close or re-open a ticket. Notification of the status change would be sent for users to follow the progress of the communication requests. In addition, a reminder would be sent to ensure that a response is received in time. The reminders could be triggered manually by the users or automatically based on defined rules. For example, a rule could be defined that “send a reminder to the receiver when there are only 5 mins left before the request is due’. The system could set up rule configuration modules for the reminder or include personalized alerts and reminders for the radiologist.

The system shown in FIG. 3 also includes a data source layer, such as the illustrative PACS 12, and/or a Radiology Information System (RIS), patient electronic records, and/or so forth, to enable access to data in these databases. To support the aforementioned functions, requisite data may be provided, which may include for example basic information of patient and study, such as patient MRN, study ID, modality, scanner, room, technologists, referral physician etc.; Staff information, such as role, specialty information of technologist and radiologists; DICOM images; Patient clinical context, such as patient history lab results; and so forth. Basic study information is used to link the communication message with a study. The information will also be presented in the worklist to provide an overview of the open communications for the receivers. Staff information is needed to identify and allow messages to be sent to the appropriate message receiver. Providing the radiologist with the patient & imaging data within this solution also has the advantage that they do not have to switch systems and can easily go back to the reading task they may have been doing in the meantime, without having to switch patients or being confused what images/data they were looking at. DICOM images and patient clinical context are used to clarity the context of the questions and also allow radiologists to review the context in the sample application.

The system shown in FIG. 3 can be a “two way device.” That is, the radiologist will have questions to the technician and the referring physician as well. One advantage of this system is also that each party does not have to remember as much information. Each party will also have more overview, as they do not have to remember questions (e.g., when someone does not pick up the phone), the status of their question and/or the patient data (e.g., patient numbers). Having two way communication can help the radiologist there too (as well as the technologist/referring physician who know what they still need to do). Additionally, the radiologist can also interrupt the technologist/referring physician.

In some embodiments, the apparatus 10 can be configured to sense specific contextual information around radiology review requests, to live update the urgency of a case ticket and the availability of the radiologist. For example, the apparatus 10 can be configured to optimize case scheduling and interruption impact. A ‘cost of interruption’ is based on the complexity of the case the radiologist is working on, as well as the engagement that the radiologist experiences while doing his/her review. This way, a technician can consider his/her reasons for interrupting with more empathy towards the radiologist based on the cost of interruption. This allows for lowering, or even excluding the impact of interruption in some cases. In one approach, the contextual adaptation weighs two sides (the urgency of the technician's request versus the cost of interruption to radiologist) and proposes a suggested follow-up action for the technician and/or the radiologist. In some embodiments, the technician is provided with a suggestive list of available radiologists that could also help solving their problem/case.

A radiologist being interrupted by a technician (or another radiologist) happens regularly in the context of an urgent case ticket or inquiry. The case urgency is determined in some embodiments by automatic assessment and/or by the technician manually assessing an urgency score for his or her request for radiology assistance. The real time “cost of interruption” is assessed automatically based on the current engagement score of the radiologist in combination with the urgency and complexity of the task that he/she is currently doing (e.g., a current radiology examination reading). These two main indications (request urgency and cost of interruption) are combined, for example as a weighted average, based on the determined significance of all the parameters mentioned. By taking both indications into consideration, a follow-up action is executed and/or suggested.

The apparatus 10 is configured as described above to perform an imaging assistance method or process 200. The database 16 stores instructions executable by the server computer 14 (and/or by the requestor electronic processing device 18 and/or the receiver electronic processing device 36) to perform the imaging assistance method or process 200. In some examples, the method 200 may be performed at least in part by cloud processing (that is, the server computer 16 may be implemented as a cloud computing resource comprising an ad hoc network of server computers).

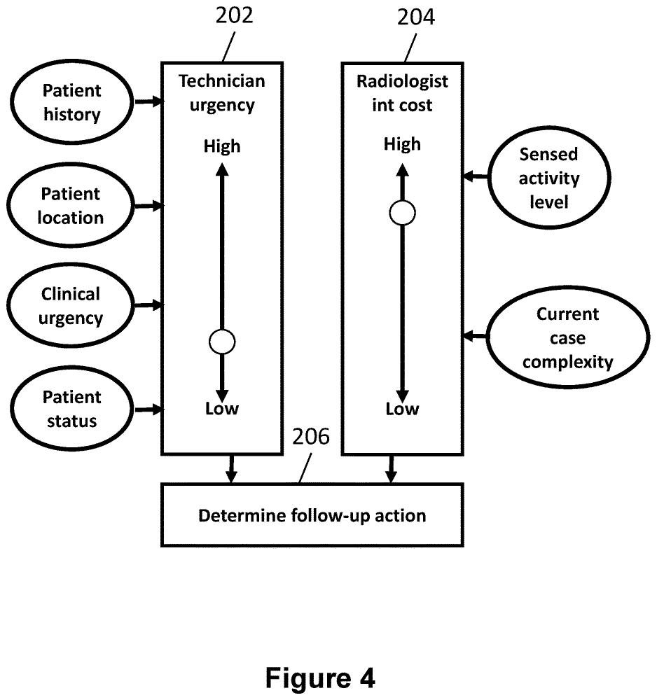

With reference to FIG. 4, and with continuing reference to FIG. 1, an illustrative embodiment of an instance of the imaging assistance method 200 is diagrammatically shown.

At an operation 202, an urgency level of the imaging examination is determined, quantified, and reported by the requestor RQ. The urgency of the imaging examination is dependent on several different factors. FIG. 4 lists four nonlimiting illustrative factors: patient history (e.g., a critically ill patient may weigh toward higher request urgency); patient location (e.g., a patient loaded in the imaging system weighs toward higher request urgency whereas a patient not scheduled for imaging today weighs toward lower request urgency); clinical urgency (e.g., an imaging session performed to assess whether a patient should immediately undergo emergency surgery weighs toward higher request urgency); and patient status (this factor could cover various circumstances such as whether the patient is an admitted hospital patient or an outpatient). For example, if the patient is still on the patient table or in the dressing room, the priority of the ticket is higher. Additionally, the type of case, its clinical urgency, and the remaining waiting list influence on the prioritization of the tickets. In some examples, the urgency changes when an answer has been found in another way, or if the patient had to leave due to another appointment. The apparatus 10 may automatically quantify urgency based on inputs from the imaging device, computer vision systems (i.e., sensing if the patient is still present), electronic medical record (EMR) (i.e., imaging request), and natural language processing (NLP) interpretation of the technicians' question in relation to an imaging knowledge database.

To perform the operation 202 of estimating the urgency of the request for radiology assistance, the apparatus 10 is configured to determine separate scores of specific parameters that are either automatically sensed or manually indicated by the technician. The patient history influencing the urgency score is filled in manually by the technician, as well as the clinical urgency score (based on the type and severity of the case), and the status and/or agenda of the patient. If the case ticket or inquiry is more urgent based on whether the patient is still on the table or in the dressing room, is automatically analyzed via a camera in the image acquisition room. All parameters have a certain level of significance that is generally determined upfront. However, this can be adjusted for every case individually. As a result of the significance and severity of every parameter, as mentioned before, a quantified urgency score is established from 1-10. These scores than are categorized into urgency levels ‘low’ (1-4), ‘medium’ (4-7), and ‘high’ (7-10).

At an operation 204, a “cost of interruption” score for the receiver RC (e.g., the radiologist in illustrative FIG. 4) is determined. The cost of interruption is determined for the radiologist operating the receiver electronic processing device 30. The cost of interruption score is based on the commitment and engagement of the receiver RC towards his/her current imaging examination reading or other current task (e.g., measured via gaze tracking patterns, pulse rate, and/or other suitable biometric sensor(s)), and the complexity and urgency of the current task. For example, if the radiologist is currently engaged in reading a complex magnetic resonance imaging examination this would weight toward a higher cost of interruption; whereas, if the radiologist is currently reading a simple chest X-ray this would weigh toward a lower cost of interruption. Therefore, the weighing of these parameters is directly connected to the “cost of distraction” of the receiver RC. The more engaged the receiver RC is in a complex image review, the higher the cost of distracting him/her (and vice versa).

The parameters considered to determine the “cost of interruption” score are the engagement of the radiologist, and the complexity & urgency of the current case that he/she is reviewing. The engagement and commitment are, in a nonlimiting illustrative embodiment employing eye tracking as the biometric sensor, quantified via measurements of the pupil dilation, the head position, and/or the body posture as can be done by an eye tracking device. The complexity and urgency score are indicated by the technician when he/she was creating the case ticket. As a result of these elements, as mentioned before, a quantified interruptability score is established, by way of nonlimiting example on a scale from 1-10. These scores than are categorized into interruption cost levels ‘low’ (1-4), ‘medium’ (4-7), and ‘high’ (7-10).

At an operation 206, a suggested follow-up action is generated by the receiver RC for the requestor RQ. To do so, the scores for urgency versus cost of interruption to determine allowed/advised communication means from the requestor RQ to the receiver RC. Depending on the balance of the two scores, the suggested follow-up action can include, for example, allowing an immediate interruption, a submission of a request ticket, or a referral to another radiologist if the case is urgent, but the preferred radiologist cannot be disturbed. The evaluation of urgency versus cost of interruption is continuous and real-time, as such the apparatus 10 may change its advice/regulation when the situation on the one and/or other side changes. Additionally, when for example the radiologist is not interruptible, the apparatus 10 provides alternatives solutions in terms of other available radiologists. Table 1 shows a list of possible suggested follow-up actions based on the urgency and the cost of interruption score.

TABLE 1

| | | Cost Of Interruption | | |
|---|---|---|---|---|
| | | Low | Medium | High |
| General | High | TECH Interrupt | TECH Consider | TECH Gets advice not |
| Case | | radiologist with urgent | interruption of radiologist | to interrupt now + |
| Urgency | | case/question | RAD Notify Rad of | referral to other |
| | | RAD Take Rad from | highly urgent | available rads |
| | | current case | case/question | RAD Add |
| | | | | case/question on top of |
| | | | | list |
| | Medium | TECH Consider | TECH Gets advice not to | TECH Gets strong |
| | | interruption of | interrupt + referral to | advice not to interrupt + |
| | | radiologist | other available rads | referral to other |
| | | RAD Notify Rad of | RAD Add case/question | available rads |
| | | medium-urgent | on list | RAD Add |
| | | case/question | | case/question on list |
| | Low | TECH Gets advice not | TECH Gets strong | TECH Not possible to |
| | | to interrupt | advice not to interrupt | interrupt + Blocked |
| | | RAD Add case/question | RAD Add case/question | communication |
| | | on list | on list | RAD Add |
| | | | | case/question on list |

In some embodiments, facial recognition or other type of user identification (e.g., login) to recognize and identify radiologists and technicians can be implemented. Identification enables connections with personal profiles regarding their preferences and agenda. This way, their personal data and properties can also play a role in this equation, allowing for a more personalized and adaptive system. As one nonlimiting illustrative example of this, each radiologist may a priori rate his or her proficiency in reading radiology examinations of different modalities (e.g., MRI, CT, PET, et cetera). This information may be used in enabling the current case complexity to be assessed in a personalized fashion, for example if the current case is an MRI reading and the radiologist (recognized by the facial recognition) has self-rated his or her proficiency in MRI readings to be low then this weighs toward the current case complexity being scored as “complex” or “highly complex”; whereas, for a different radiologist who has self-rated his or her proficiency in MRI readings to be high then this would instead weigh toward the current case complexity being scored as “low” or “moderate”.

In some embodiments, emotional state sensing based on the biometric measurements of the radiologist (e.g., facial expression, galvanic skin response, HRV, etc.) can be implemented. This also plays a role in the interruptability of the radiologist. When a radiologist is very stressed and frustrated, the “cost of interruption” is thought to be higher than when he/she feels calm and neutral. This results in an empathic system with even more personalized and collaborative capabilities.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by an electronic processor to perform a method of requesting radiology assistance, the method comprising:
providing, on a display device of a requestor electronic processing device operable by a requestor, a requestor graphical user interface (GUI);
receiving one or more inputs from the requestor to fill in fields in the requestor GUI to generate an assistance request for radiology assistance;
transmitting the assistance request for radiology assistance to a receiver electronic processing device operable by a radiologist;
providing, on a display device of the receiver electronic processing device, a receiver GUI showing a list of assistance requests for radiology assistance; and
tracking and labelling a status of each assistance request for radiology assistance in the list.

2. The non-transitory computer readable medium of claim 1, wherein the fields in the requestor GUI include at least:
an addressee field configured to receive an addressee as a specific radiologist or a radiologist role;
a field for assigning a priority on a predefined prioritization schedule; and
a field for attached documentation or links to documentation.

3. The non-transitory computer readable medium of claim 1, wherein the list of assistance requests displayed on the receiver GUI includes a worklist of the requests addressed to the receiver based on one or more criterion including at least priority or a reply-by time.

4. The non-transitory computer readable medium of claim 1, wherein the receiver GUI includes a field for a communication pathway between the receiver and each requestor who submitted an assistance request.

5. The non-transitory computer readable medium of claim 1, wherein the receiver GUI and/or the requestor GUI includes a field for updating the status of the assistance request.

6. The non-transitory computer readable medium of claim 1, wherein the fields of the requestor GUI include a field for attaching a clinical image that is the subject of the assistance request and via which the requestor can annotate the clinical image, the annotated clinical image being entered into the field for attaching the clinical image.

7. The non-transitory computer readable medium of claim 1, wherein the method further includes:
establishing a natural communication pathway between the requestor and the receiver.

8. The non-transitory computer readable medium of claim 1, wherein the method further includes:
generating the assistance request using an artificial intelligence (AI) component.

9. The non-transitory computer readable medium of claim 8, wherein the AI component is configured to:
analyze a computer-aided diagnostic (CADx) feature of an imaging device; and
generating the assistance request based on the analysis of the CADx feature.

10. The non-transitory computer readable medium of claim 8, wherein the AI component is configured to:
analyze a clinical image generated by an imaging device; and
generating the assistance request based on the analysis of the clinical image.

11. The non-transitory computer readable medium of claim 1, wherein the method further includes:
monitoring a schedule of upcoming radiology examinations including detecting an upcoming radiology examination with an incomplete examination card that requires information from a radiologist to complete the examination card; and
generate a request for the requestor to provide the information required to complete the examination card before a beginning of an imaging examination.

12. The non-transitory computer readable medium of claim 1, wherein the tracking and labelling of the status of each assistance request for radiology assistance in the list includes:
estimating an urgency of each assistance request for radiology assistance in the list;
estimating an interruption cost of interrupting a radiologist operating the receiver electronic processing device; and
updating the status of each assistance request for radiology assistance in the list based on the urgency score estimated for that assistance request and the estimated interruption cost.

13. The non-transitory computer readable medium of claim 12, wherein the interruption cost is estimated at least in part based on biometric sensor data acquired of the radiologist operating the receiver electronic processing device.

14. The non-transitory computer readable medium of claim 12, wherein the interruption cost is estimated at least in part based on a complexity of a current task being performed by the radiologist operating the receiver electronic processing device.

15. An assistance request method, the method comprising:
receiving one or more inputs from a requestor to fill in fields in a requestor graphical user interface (GUI) displayed on a display device of a requestor electronic processing device (18) operable by a requestor to generate an assistance request for radiology assistance;
transmitting the assistance request for radiology assistance to a receiver electronic processing device operable by a radiologist; and
tracking and labelling a status of the assistance request for radiology assistance on a receiver GUI provided on a display device of the receiver electronic processing device.

16. The assistance request method of claim 15 wherein the tracking and labelling of the status of each assistance request for radiology assistance in the list includes iteratively:
estimating an urgency of the assistance request for radiology assistance;
estimating an interruption cost of interrupting a radiologist operating the receiver electronic processing device; and
updating the status of the assistance request for radiology assistance based on the urgency score and the estimated interruption cost.

17. An assistance request method, the method comprising:
determining an urgency of a medical imaging examination performed by a requestor;
determining an interruption score of a radiologist from whom assistance is being requested by the requestor; and
determining at least one follow-up action for the radiologist to provide to the requestor based on the determined urgency and the determined interruption score.

18. The method of claim 17, wherein determining the interruption score includes:
sensing one or more biometric parameters of the radiologist; and
determining the interruption score based on the sensed biometric parameters.

19. The method of claim 17, wherein determining the urgency includes:
determining a complexity of the medical imaging examination; and
determining the urgency based on the determined complexity.

20. The method of claim 17, wherein determining the at least one follow-up action includes:
weighting the urgency and the interruption score to determine one or more permitted or recommended communication pathways.

21. The method of claim 17, wherein the method further includes:
identifying at least one of the radiologist and the requestor using facial recognition, the at least one follow-up action being determined further based on the identifying.

22. The method of claim 17, wherein the at least one follow-up action includes allowing an immediate interruption, a submission of a request ticket, or a referral to another radiologist if the case is urgent, but the preferred radiologist cannot be disturbed.
